(19) United States
(12) Patent Application Publication
(10) Pub. No.: US 2020/0230382 A1
(43) Pub. Date: Jul. 23, 2020

(54) URETHRAL DEVICE

(71) Applicant: Madeleine RAMSTEDT, Umeå (SE)

(72) Inventor: Jost Siebert, Søborg (DK)

(21) Appl. No.: 16/500,596

(22) PCT Filed: Apr. 4, 2018
(86) PCT No.: PCT/SE2018/000010

§ 371 Date: Oct. 3, 2019

(30) Foreign Application Priority Data
Apr. 4, 2017 (SE) 1750414-3

(51) Int. Cl. A61M 27/00; A61M 25/00; A61F 2/04; A61M 25/09; A61M 25/04

(52) U.S. Cl. CPC A61M 27/008; A61M 25/0017; A61F 2/04; A61M 25/007; A61M 25/09; A61F 2210/0014; A61M 2210/1089; A61M 2210/1085; A61M 2202/0496; A61F 2002/047; A61M 2205/0266; A61M 25/04

(57) ABSTRACT

An indwelling urethral device is described that includes an elongated tubular member extending along a longitudinal axis (L) from a distal inlet end portion, via an intermediate portion, to a proximal outlet end portion, the tubular member defining a lumen extending along the longitudinal axis between the distal inlet end portion and the proximal outlet end portion, the distal end portion of the elongated tubular member comprising a plurality of urine inlet openings, at least a part of the distal end portion being preconfigured to be reversibly transformable between a linear state for passage through the urethra when inserted there through and a preconfigured coiled state for anchored receipt within the bladder in the use position. The plurality of urine inlet openings is provided on the interior spiral section and urine inlet openings are absent on the exterior spiral section.

### TECHNICAL FIELD

The present disclosure relates to an indwelling urethral device, such as an indwelling urethral stent, for use in drainage of urine from a bladder when the device in a use position is indwelling in a patient. The present invention also relates to an intermittent urethral catheter for use in drainage of urine from a bladder of a patient.

### BACKGROUND

Catheters have been used for a very long time as a device for treating diseases or performing surgical procedures. A catheter is usually a thin tube, also referred to as stent, which may be used to inject or drain fluids and/or liquids from the body, such as for draining blood or for injecting medicine into the body.

A common use of a catheter is in the form of a urethral catheter, where a thin hollow tube is inserted into the urinary bladder via the urethra in order to drain the bladder of urine that has been collected in the urinary bladder.

There are two general usages of urinary catheters, where the first use is intermittent catheterization and the second use is permanent catheterization.

Intermittent catheterization is where an intermittent catheter is inserted into the urethra to drain the bladder, and after use the catheter is removed from the body. An intermittent catheter is a relatively stiff catheter having a hydrophilic or gel coating for lubrication and allows the users to perform the insertion on their own.

Permanent catheterization is where a relatively soft and flexible catheter is inserted into the body, and is anchored inside the body, so that the catheter is maintained in place for a significant period, such as days or weeks, in order to have permanent drainage of the bladder and to reduce the need of multiple insertions of catheters for healthcare personnel. This type of catheter is referred to as an indwelling catheter.

Urinary catheters of the indwelling type are generally provided with an inflatable retention member disposed at a distal inlet end portion of the catheter to anchor the catheter in the bladder, the catheter including at least one drainage channel to drain urine from the bladder and at least one inflation channel to inflate the retention member (e.g. with sterile water). The proximal end of a urinary catheter of the indwelling type furthermore generally includes at least two ports in fluid communication with the two channels, a first port connected to the drainage channel and having an interface for fittings for drainage and taking of samples and a second port which is connected to the inflation channel via a valve to ensure that the inflation fluid remains within the channel and in the retention member once filled. The tip of the distal inlet end portion of a conventional urinary catheter of the indwelling type extends beyond the location of the retention member into the bladder and includes one or more apertures/urine inlet openings to drain urine from the bladder.

A consequence of the retention member being positioned below the urine inlet opening is a risk for incomplete emptying of the bladder.

Indwelling urinary catheters may comprise parts extending externally from the user's body. In this type of urinary catheters, the proximal end of the catheter tube is adapted to at least partly extend outside the user's body during use.

Alternatively, indwelling urinary catheters may be completely internally housed within the user's body during use. This type of urinary catheters, also referred to as urinary stents or prostatic stents, may be inserted into the urethra to prevent obstruction of the urethra and thereby drain urine from the bladder.

WO 2007/001978 A1 relates to an indwelling urethral device including a prostatic urethral stent body and a urethral anchoring element. The prostatic urethral stent body includes a preconfigured end portion for anchored receipt within a bladder with the urethral anchoring element extending from the prostatic urethral stent body via a linkage. The preconfigured end portion is formed as a “rolled-up” or spiraled free end which is either aligned with or axially orthogonal in relation to an axis of elongation of the stent body. A plurality of urine inlet openings extends through the preconfigured portion.

A problem with indwelling and intermittent urethral devices is that the bladder may be damaged due to the under-pressure created during emptying of the bladder. The bladder wall may even be sucked into the urine inlet openings and thereby damaged.

Another problem with indwelling urethral device is that they may cause bacterial infections in the urinary tract.

### SUMMARY

An object of the present disclosure is to alleviate one or more of the above problems and to provide an improved indwelling urethral device, such as an indwelling urethral catheter or an indwelling urethral stent, or alternatively an intermittent urethral catheter.

The present disclosure relates to an indwelling urethral device for use in drainage of urine from a bladder when the device in a use position is indwelling in a patient. The urethral device comprises an elongated tubular member extending along a longitudinal axis (i.e. the axis of elongation) from a distal inlet end portion via an intermediate portion to a proximal outlet end portion. The tubular member defines a lumen extending along the longitudinal axis between the distal inlet end portion and the proximal outlet end portion, the distal end portion of the elongated tubular member comprising a plurality of urine inlet openings, the proximal end portion of the elongated tubular member having a drainage outlet, and the lumen of the elongated tubular member provides fluid communication between the plurality of urine inlet openings and the drainage outlet. At least a part of the distal end portion is preconfigured to be reversibly transformable between a linear state for passage through the urethra when inserted there through and a preconfigured coiled state for anchored receipt within the bladder in the use position. The preconfigured coiled state of the distal end portion forms a spiral emanating from a point on the longitudinal axis of the elongated tubular member and the spiral extends in at least a first and a second direction, the second direction being orthogonal to the first direction and each of the first and second direction being axially transverse to the longitudinal axis of the elongated tubular member. The spiral comprises an interior spiral section facing inwards the spiral and an exterior spiral section facing outwards away from the spiral. The plurality of urine inlet openings is provided on the interior spiral section and urine inlet openings are absent on the exterior spiral section.

The distal end portion or said part of the distal end portion, which is preconfigured to be reversibly transformable between the linear state and the preconfigured coiled state, is predisposed to revert to the preselected coiled configuration when the urethral device in the use position is indwelling.

When the preconfigured distal end portion or said preconfigured part of the distal end portion is in the linear state, the distal end portion has a straightened, elongated form.

In particular, the spiral may be a two-dimensional spiral extending in a plane defined by the first and the second directions. However, the spiral may alternatively be the configured as a three-dimensional spiral extending in three directions, such as following a conic structure.

The preconfigured coiled state of the distal end portion forms a spiral emanating from a point on the longitudinal axis of the elongated tubular member and the spiral extends in at least a plane defined by a first and a second direction, where the second direction is orthogonal to the first direction and each of the first and second direction is axially transverse to the longitudinal axis of the elongated tubular member which means that the preconfigured coiled state of the distal end portion provides a disc-like bladder anchor having a lateral extent substantially perpendicular to the longitudinal axis of the elongated tubular member. The preconfigured coiled state of the distal end portion enables distal anchoring in the bladder and prohibits movement of the indwelling urethral device proximally from the bladder when the device is in the use position.

The fact that the spiral includes the plurality of urine inlet openings has the effect of allowing the urine inlet opening of the indwelling urethral device to be located in close proximity to the urine bladder neck, which improves emptying of the bladder and prevents post void residual urine from accumulating near the bladder neck. Thus, the positioning of the urine inlet openings in close proximity to the lower portion (bottom) of the bladder ensures substantially complete emptying of the bladder.

Moreover, due to the fact that the plurality of urine inlet openings is provided on the interior spiral section and that urine inlet openings are absent on the exterior spiral section, there will be a reduced risk for bladder damage due to the under-pressure created during emptying of the bladder.

The plurality of urine inlet openings may be in the form of eyelets, each eyelet having a maximum length or width, such as diameter, within the range of from 2 to 7 mm, such as within the range of from 3 to 5 mm.

The plurality of urine inlet openings may have a circular shape or an oval shape. The plurality of urine inlet openings may be spaced such that there is a distance of at least 2 mm between neighboring urine inlet openings. In particular, the distance between neighboring urine inlet openings may be within the range of from 2 to 20 mm.

The interior spiral section may comprise, for instance, within the range of from 3 to 9 urine inlet openings.

In particular, the spiral of the indwelling urethral device as disclosed herein may revolve around a point on the longitudinal axis of the elongated tubular member such that coiled distal end portion moves progressively farther from said point in such a way that the spiral extends laterally, in relation to the longitudinal axis of the elongated tubular member, around said point on the longitudinal axis of the elongated tubular member.

In particular, the indwelling urethral device as disclosed herein may be an indwelling urethral stent configured to be situated entirely inside the patient when the stent in the use position is indwelling.

In embodiments where the indwelling urethral device is an indwelling urethral stent, the indwelling urethral stent may further comprise a retention member for retaining the urethral device within the urethra. The retention member is to be disposed externally of the bladder and in the urethra, when the urethral stent in the use position is indwelling. The retention member may be provided at or in proximity to the proximal outlet end portion. The retention member enables proximal anchoring in the urethra and prohibits movement of the indwelling catheter distally when the urethral stent is in the use position.

The retention member may be provided by a part of the proximal end portion preconfigured to be transformable between a linear state for passage through the urethra when inserted there through and a twisted preconfigured state for retention thereof within the urethra when the urethral stent in the use position is indwelling.

Alternatively, the retention member may be provided by a part of the proximal end portion preconfigured to be transformable between a linear state for passage through the urethra and a compressed preconfigured state forming a bump on the elongated tubular member for retention thereof within the urethra when the urethral stent in the use position is indwelling. In this embodiment, the part of the proximal end portion which provides the retention member bulges outwardly when the retention member is in the compressed preconfigured state.

Moreover, in both these embodiments, the part of the proximal end portion which provides the retention member has a flush outer surface when the retention member is in the linear state.

The urethral device as disclosed herein may be a urethral catheter. The urethral catheter may comprise a drainage port at the proximal outlet end portion, the drainage port being provided with means for connecting the drainage port to a urine collection bag or to a sampling device. The urethral catheter as disclosed herein may alternatively be an intermittent urinary catheter, for use in drainage of urine from a bladder when the urethral catheter in a use position is intermittently inserted in a patient.

The distal inlet end portion may be provided with an end opening. This may facilitate insertion of the urethral device by means of a guide wire/inserter. The end opening may have a diameter within the range of from 2 to 7 mm, such as within the range of from 3 to 5 mm.

The indwelling urethral device as disclosed herein may be a single lumen urethral device, such as a single lumen urethral stent.

Due to the fact that no inflation channel is needed in the urethral device as disclosed herein, the cross-section of the lumen of the elongated tubular member of the urethral device as disclosed herein may be larger than the cross-section of the drainage channel of a urethral device comprising also an inflation channel. A wider lumen may provide a more efficient drainage as well as a reduced risk for blockage of the lumen due to biofilm formation on the interior walls of the lumen.

The distal inlet end portion, the intermediate portion and the proximal outlet end portion of the indwelling urethral device may be formed as an integral unit.

In particular, the indwelling urethral device may be moulded as an integral unit using a synthetic material, such as biocompatible silicone.

In particular, the indwelling urethral device as disclosed herein may be configured to be situated entirely inside the patient when the urethral device in the use position is indwelling. Due to the fact that no part of the indwelling urethral device is positioned outside the patient when the urethral device in the use position is indwelling, has the effect that it will be more difficult for bacteria to enter the urinary tract and the risk for bacterial infections may be reduced.

In particular, the elongated tubular member may have a varying wall thickness along the longitudinal axis such that a section of the intermediate portion of the elongated tubular member has a wall thickness which is less than the wall thickness of any adjacent section of the elongated tubular member. The thin-walled section of the intermediate portion of the elongated tubular member may be configured to be positioned in proximity to urethral sphincter (also referred to as external sphincter) when the urethral device in the use position is indwelling.

The thin-walled section of the intermediate portion of the elongated tubular member allows for substantially complete closure of the lumen of the urethral device by the urethral sphincter. Thus, the normal function of the urethral sphincter is kept and the patient maintains physiologically normal control of the urination.

In particular, each of the distal end portion and the proximal end portion of the elongated tubular member may have a first wall thickness and at least a section of the intermediate portion of the elongated tubular member may have a second wall thickness less than the first wall thickness of the distal end portion and the proximal end portion.

The first wall thickness may be within the range of from 0.5 to 2.5 mm.

The second wall thickness may be within the range of from 0.2 to 1.5 mm.

The indwelling urethral device as disclosed herein may have an outer diameter of the elongated tubular member within the range of from about 5 to about 10 mm.

The indwelling urethral device as disclosed herein may have a length within the range of from about 6 to about 22 cm along the longitudinal axis of the elongated tubular member when the distal end portion is in the linear state.

The indwelling urethral device as disclosed herein may have a length within the range of from about 3 to about 10 cm along the longitudinal axis of the elongated tubular member when the distal end portion is in the preconfigured coiled state.

Thus, the preconfigured part of the distal end portion of the elongated tubular member may be within the range of from about 30 to 50%, such as from 40 to 50%, of the total length of the elongated tubular member.

During insertion of the urethral device into the urethra it is important that the urethral device slides easily through the urethra without exposing the urethral wall to any risk of damage. It is also important that the urethral device in the use position stays in place and is capable of following the urethra without inducing discomfort to the patient using it.

In particular, the elongated tubular member may be formed at least partially from a pliable, biocompatible material, such silicone.

At least the preconfigured part of the distal end portion may be formed at least partially from a memory-shape material, such as a shape-memory polymeric (SMP) material, a shape-memory alloy (SMA) material, or a shape-memory composite material.

In certain embodiments, the entire elongated tubular member may be formed at least partially from a memory-shape material, such as a shape-memory polymer, shape-memory alloy or a shape-memory composite material.

Memory-shape materials are materials that are capable of returning from a deformed state (temporary shape) to their original (permanent) shape induced by an external stimulus, such as temperature increase or mechanical stress.

Examples of suitable shape-memory polymers are polyurethane.

Examples of suitable shape-memory alloys are Nitional (alloy of nickel and titanium).

Furthermore, the distal inlet end portion may comprise a so-called angular Tiemann tip (also called ball tip) in order enable the prostate gland to be easier circumnavigated and thereby facilitate insertion of the urethral device into the urethra.

The present disclosure also relates to a urethral device assembly comprising a urethral device as disclosed herein and a reinforcing guide wire adapted to be inserted coaxially into the lumen of the tubular member of the urethral device during insertion of the urethral device through the urethra.

The reinforcing guide wire facilitates the insertion of the urethral device by providing an inner support to the soft urethral device. Thus, having the guide wire inserted coaxially into the lumen of the urethral device ensures that the urethral device has enough rigidity to allow insertion into the urethra.

The guide wire may be attached to an insertion device during the insertion procedure as well known to persons skilled in the art.

Moreover, by applying a pushing force to the reinforcing guide wire, the preconfigured part of the distal end portion is forced to its (temporarily) linear state to enable passage of the urethral device through the urethra. When the reinforcing guide wire is removed, the preconfigured part of the distal end portion reverts to its preconfigured coiled state to enable anchored receipt within the bladder in the use position.

The guide wire may either be of the type that is inserted and designed to navigate the urethra to reach the bladder. Once the tip of the guide wire reaches the bladder, it acts as a guide that the urethral device can follow. The guide wire may also be of the inserter type that is inserted into the lumen of the tubular member of the urethral device prior insertion into the urethra to force the urethral device into its temporarily linear state.

### BRIEF DESCRIPTION OF DRAWINGS

FIG. 1 illustrates an embodiment of an indwelling urethral device according to the present disclosure. The urethral device is shown in the linear state as well as in the preconfigured coiled state.

FIG. 2 illustrates the coiled distal end portion of the indwelling urethral device of FIG. 1.

FIG. 3 is a cross-section taken along the longitudinal axis of the elongated tubular member of the urethral device of FIG. 2.

FIG. 4 illustrates the indwelling urethral device of FIG. 1 in use position when the urethral device is indwelling.

FIG. 5 illustrates a first type of retention member which is part of the indwelling urethral device of FIG. 1. This figure also illustrates the function of a thin-walled intermediate section.

FIG. 6 illustrates a second type of retention member of an indwelling urethral device as disclosed herein as well as the function of a thin-walled intermediate section.

FIG. 7 illustrates an indwelling urethral device as disclosed herein having a so-called Tiemann tip.

FIG. 8 illustrates the urethral device, in the preconfigured coiled state, according to the present disclosure provided with a drainage port.

FIG. 9 illustrates the urethral device in FIG. 8 in the linear state.

### DETAILED DESCRIPTION

It is to be understood that the drawings of the urethral devices shown are schematic and that individual components are not necessarily drawn to scale.

As used herein, “distal portion” refers to the portion of the urethral device furthest away from the medical operator inserting the urethral device in the urethra of a patient. Thus, the distal portion of the urethral device is the portion which is configured to be positioned in the bladder of the patient when in use.

As used herein, “proximal portion” refers to the portion of the urethral device closest to the medical operator inserting the urethral device in the urethra of a patient. Thus, the proximal portion of the urethral device is the portion which is configured to be positioned externally of the bladder of the patient when the urethral device is in use (indwelling). The proximal portion of the urethral device may be positioned in the urethra or outside the patient when the urethral device is in use (indwelling).

As used herein, the term “urinary catheter” refers to a device including a thin tube which is adapted to be inserted into the urethra and the urinary bladder to void the bladder of urine that has been collected in the urinary bladder. The term urinary catheter includes both urinary catheters partly extending externally from the user's body and urinary catheters which are adapted to be completely internally housed within the user body during use. The latter type of urinary catheters may also be referred to as urinary stents or prostatic stents.

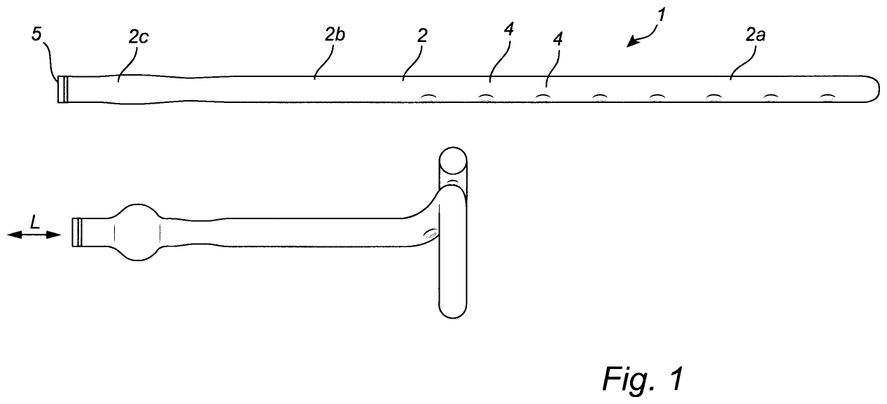

FIG. 1 illustrates an embodiment of an indwelling urethral device 1, in this case an indwelling urethral stent, for use in drainage of urine from a bladder when the urethral device in a use position is indwelling in a patient. The urethral device is a single lumen urethral device, which comprises an elongated tubular member 2 extending along a longitudinal axis L from a distal inlet end portion 2a via an intermediate portion 2b to a proximal outlet end portion 2c. In the illustrated embodiment, the distal inlet end portion, the intermediate portion and the proximal outlet end portion of the indwelling urethral device is formed as an integral unit.

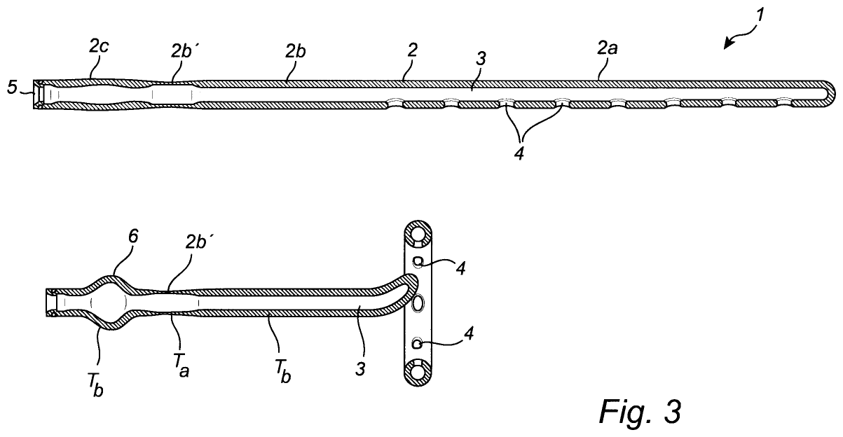

As shown in FIG. 3, the tubular member 2 defines a lumen 3 extending along the longitudinal axis L between the distal inlet end portion 2a and the proximal outlet end portion 2c.

The distal end portion 2a of the elongated tubular member 2 comprises a plurality of urine inlet openings 4.

The proximal end portion 2c of the elongated tubular member 2 comprises a drainage outlet 5.

The lumen 3 of the elongated tubular member 2 provides fluid communication between the plurality of urine inlet openings 4 and the drainage outlet 5.

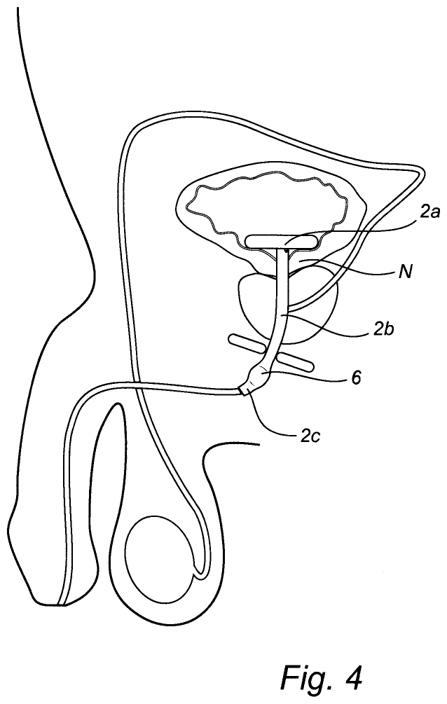

At least a part of the distal end portion 2a is preconfigured to be reversibly transformable between a linear state (the upper figure of FIG. 1) for passage through the urethra when inserted there through and a preconfigured coiled state (the lower figure of FIG. 1) for anchored receipt within the bladder in the use position as shown in FIG. 4.

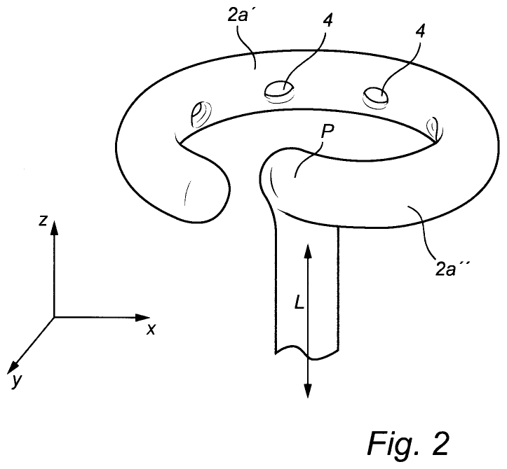

As illustrated in more detail in FIG. 2, the preconfigured coiled state of the distal end portion 2a forms a spiral emanating from a central point P on the longitudinal axis L of the elongated tubular member 2 and the spiral extends in at least a first direction x and a second direction y, the second direction y being orthogonal to the first direction x and each of the first and second direction x, y being axially transverse to the longitudinal axis L of the elongated tubular member 2.

The spiral comprises an interior spiral section 2a′ facing inwards the spiral and an exterior spiral section 2a″ facing outwards away from the spiral. The plurality of urine inlet openings 4 is provided on the interior spiral section 2a′ and urine inlet openings are absent on the exterior spiral section 2a″.

As seen in FIG. 3, the preconfigured coiled state of the distal end portion 2a provides a disc-like bladder anchor having a lateral extent substantially perpendicular to the longitudinal axis L of the elongated tubular member 2. As further seen, the urine inlet openings 4 of the indwelling urethral device 1 are located in close proximity to the urine bladder neck N.

In the illustrated embodiment, there are about 8 urine inlet openings 4, each oval-shaped opening 4 having a maximum length within the range of from 2 to 7 mm. The urine inlet openings 4 are spaced such that there is a distance within the range of from about 2 to 20 mm, such as from about 5 to 18 mm, between neighboring urine inlet openings 4.

As shown in FIG. 3, the elongated tubular member 2 has a varying wall thickness T along the longitudinal axis L such that a section 2b′ of the intermediate portion 2b of the elongated tubular member 2 has a wall thickness Tₐ which is less than the wall thickness T_{b} of any adjacent section of the elongated tubular member 2. The thin-walled section 2b′ of the intermediate portion 2b of the elongated tubular member 2 is configured to be positioned in proximity to urethral sphincter S (also referred to as external sphincter) when the urethral device in the use position is indwelling.

As shown in FIG. 3, the thin-walled section 2b′ of the intermediate portion 2b of the elongated tubular member 2 allows for substantially complete closure of the lumen 3 of the urethral device 1 by the urethral sphincter S.

The first wall thickness Tₐ may be within the range of from 0.2 to 1.5 mm.

The second wall thickness T_{b} may be within the range of from 0.5 to 2.5 mm.

The length l of the elongated tubular member of the illustrated urethral device in the linear state may be within the range of from about 6 to 22 cm, such as about 15 to 20 cm.

The outer diameter d of elongated tubular member is about 5-10 mm.

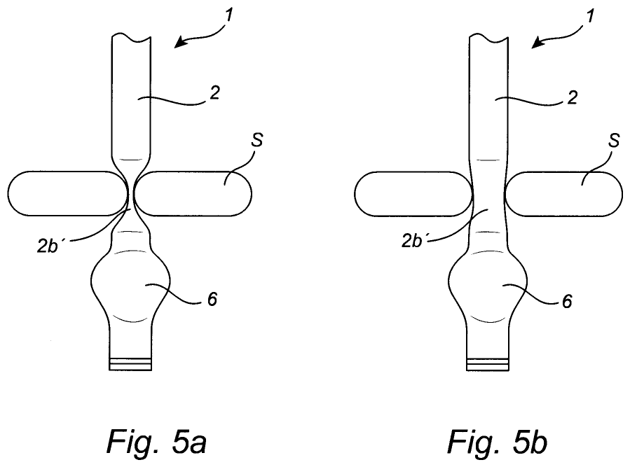

FIG. 5 illustrates a first type of a retention member 6 for retaining the urethral device 1 within the urethra. The retention member 6 is to be disposed externally of the bladder and in the urethra, when the urethral device 1 in the use position is indwelling. The retention member 6 enables proximal anchoring in the urethra and prohibits movement of the indwelling urethral device 1 distally when the urethral device 1 is in the use position. The retention member 6 is provided by a part of the proximal end portion 2c which has been preconfigured to be transformable between a linear state for passage through the urethra and a compressed preconfigured state forming a outwardly bulging bump on the elongated tubular member 2 for retention thereof within the urethra when the urethral device 1 in the use position is indwelling.

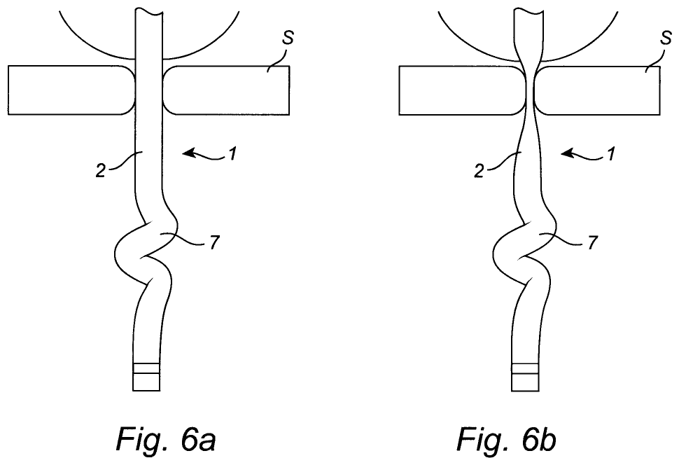

FIG. 6 illustrates another type of a retention member 7 for retaining the urethral device 1 within the urethra. In this embodiment, the retention member 7 is provided by a part of the proximal end portion 2c preconfigured to be transformable between a linear state for passage through the urethra when inserted there through and a twisted preconfigured state for retention thereof within the urethra when the urethral device 1 in the use position is indwelling.

The herein disclosed urethral device may be inserted in the urethra using a reinforcing guide wire. The guide wire is inserted coaxially into the lumen 3 of the tubular member 2 of the urethral device 1 during insertion of the urethral device 1 through the urethra.

By applying a pushing force to the reinforcing guide wire, the preconfigured part of the distal end portion 2a is forced to a temporarily linear state to enable passage of the urethral device 1 through the urethra. When the reinforcing guide wire is removed, the preconfigured part of the distal end portion 2a reverts to its preconfigured coiled state to enable anchored receipt within the bladder in the use position.

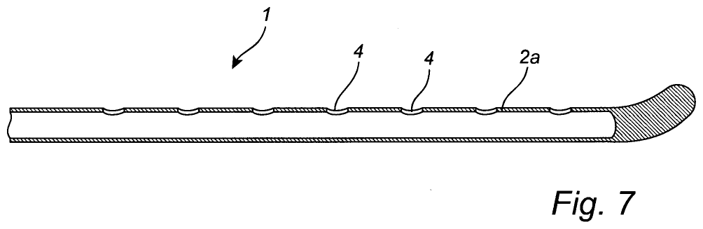

Moreover, as illustrated in FIG. 7, the distal inlet end portion 2a may comprise a so-called angular Tiemann tip (also called ball tip) in order enable the prostate gland to be easier circumnavigated and thereby facilitate insertion of the urethral device 1 into the urethra.

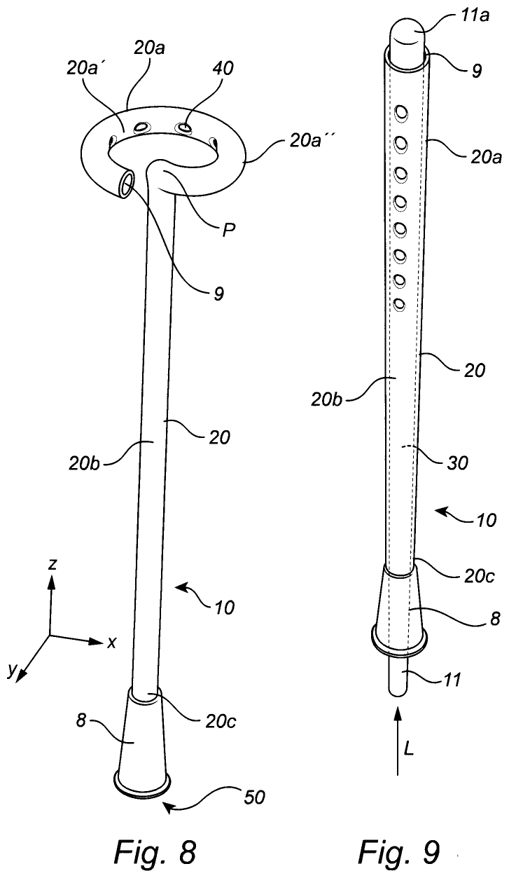

FIGS. 8 and 9 illustrate an embodiment of an urethral device 10 for use in drainage of urine from a bladder when the urethral device in a use position is in a patient. The urethral device 10 may be either an indwelling or an intermittent catheter. The urethral device is a single lumen urethral device, which comprises an elongated tubular member 20 extending along a longitudinal axis L from a distal inlet end portion 20a via an intermediate portion 20b to a proximal outlet end portion 20c. In the illustrated embodiment, the distal inlet end portion, the intermediate portion and the proximal outlet end portion of the urethral device is formed as an integral unit. The proximal outlet end portion 20c includes a drainage outlet 50 and a drainage port 8. The drainage port 8 may be provided with means for connecting the drainage port 8 to a urine collection bag or to a sampling device.

The distal inlet end portion 20a is provided with an end opening 9. As illustrated in FIG. 9, the urethral device 10 may be inserted in the urethra using a reinforcing guide wire 11. The guide wire 11 is inserted coaxially into the lumen 30 of the tubular member 20 of the urethral device 10 during insertion of the urethral device 1 through the urethra. FIG. 9 thus illustrates the urethral device 10 provided in a linear state for passage through the urethra. The end opening 9 provided in the distal inlet end portion 20a may be provided to allow easy insertion of the urethral device 10 by means of a guide wire 11. The guide wire 11 may be provided with a more flexible material in the end tip 11a of the guide wire 11 intended to protrude out from the end opening 9 during insertion through the urethra. The urethral device 10, the guide wire 11 or the end tip 11a may be manufactured using a synthetic material such as biologically compatible silicone. Additionally, at manufacturing of urethral devices 1,10 these may be moulded as an integral unit and there after sequentially cut to have a desired length, such manufacturing method provides the urethral devices 1,10 with an end opening 9 at the distal inlet end portion 2a,20a of the elongated tubular member 2,20 and a drainage outlet 5,50 in a proximal outlet end portion 2c,20c.

FIG. 8 illustrates the urethral device 10 in a preconfigured coiled state for anchored receipt within the bladder in the use position. The preconfigured coiled state of the distal end portion 20a forms a spiral emanating from a point P on the longitudinal axis L of the elongated tubular member 20 and the spiral extending in at least a first x and a second direction y. The second direction is orthogonal to the first direction and each of the first and second direction x, y are axially transverse to the longitudinal axis L of the elongated tubular member 20. The spiral comprises an interior spiral section 20a′ facing inwards the spiral and an exterior spiral section 20a″ facing outwards away from the spiral. The plurality of urine inlet openings 40 is provided on the interior spiral section 20a′ and urine inlet openings are absent on the exterior spiral section 20a″

1. An indwelling urethral device for use in drainage of urine from a bladder when the urethral device in a use position is indwelling in a patient, the urethral device comprising
an elongated tubular member extending along a longitudinal axis (L) from a distal inlet end portion, via an intermediate portion, to a proximal outlet end portion, the tubular member defining a lumen extending along the longitudinal axis between the distal inlet end portion and the proximal outlet end portion,
the distal end portion of the elongated tubular member comprising a plurality of urine inlet openings,
the proximal end portion of the elongated tubular member having a drainage outlet,
the lumen of the elongated tubular member providing fluid communication between the plurality of urine inlet openings and the drainage outlet,
at least a part of the distal end portion being preconfigured to be reversibly transformable between a linear state for passage through the urethra when inserted there through and a preconfigured coiled state for anchored receipt within the bladder in the use position, the preconfigured coiled state of the distal end portion forming a spiral emanating from a point (P) on the longitudinal axis (L) of the elongated tubular member and the spiral extending in at least a first (x) and a second direction (y), the second direction being orthogonal to the first direction and each of the first and second direction (x, y) being axially transverse to the longitudinal axis (L) of the elongated tubular member, the spiral comprising an interior spiral section facing inwards the spiral and an exterior spiral section facing outwards away from the spiral,
characterized in that
the plurality of urine inlet openings are provided on the interior spiral section and urine inlet openings are absent on the exterior spiral section.

2. An indwelling urethral device according to claim 1, wherein the spiral revolves around said point (P) on the longitudinal axis (L) of the elongated tubular member.

3. An indwelling urethral device according to claim 1, wherein the urethral device is an urethral catheter.

4. An indwelling urethral device according to claim 3, wherein the urethral catheter comprises a drainage port at the proximal outlet end portion and the drainage port is provided with means for connecting the drainage port to a urine collection bag or to a sampling device.

5. An indwelling urethral device according to claim 1, wherein the urethral device is an urethral stent.

6. An indwelling urethral device according to claim 5, further comprising a retention member for retaining the urethral stent within the urethra, to be disposed externally of the bladder and in the urethra, when the urethral stent in the use position is indwelling, the retention member being provided at or in proximity to said proximal outlet end portion.

7. An indwelling urethral device according to claim 6, wherein the retention member is provided by a part of the proximal end portion preconfigured to be transformable between a linear state for passage through the urethra when inserted there through and a twisted preconfigured state for retention thereof within the urethra when the urethral stent in the use position is indwelling.

8. An indwelling urethral device according to claim 6, wherein the retention member is provided by a part of the proximal end portion preconfigured to be transformable between a linear state for passage through the urethra and a compressed preconfigured state forming a bump on the elongated tubular member for retention thereof within the urethra when the urethral stent in the use position is indwelling.

9. An indwelling urethral device according to claim 1, wherein the indwelling urethral device is a single lumen urethral device.

10. An indwelling urethral device according claim 1, wherein the distal inlet end portion, the intermediate portion and the proximal outlet end portion of the indwelling urethral device is an integral unit.

11. An indwelling urethral device according to claim 1, wherein the elongated tubular member has a varying wall thickness (T) along the longitudinal axis (L) such that a section of the intermediate portion of the elongated tubular member has a wall thickness (Tₐ) which is less than the wall thickness (T_{b}) of any adjacent section of the elongated tubular member, said section of the intermediate portion of the elongated tubular member being configured to be positioned in proximity to urethral sphincter (S) when the device in the use position is indwelling.

12. An indwelling urethral device according to claim 1, wherein the elongated tubular member is formed at least partially from a shape memory material.

13. A device assembly comprising an indwelling urethral device according to claim 1 and a guide wire adapted to be inserted coaxially into the lumen of the tubular member of the urethral device during insertion of the urethral device through the urethra.

14. An intermittent urethral catheter for use in drainage of urine from a bladder when the urethral catheter in a use position is intermittently inserted in a patient, the urethral device comprising
an elongated tubular member extending along a longitudinal axis (L) from a distal inlet end portion, via an intermediate portion, to a proximal outlet end portion, the tubular member defining a lumen extending along the longitudinal axis between the distal inlet end portion and the proximal outlet end portion,
the distal end portion of the elongated tubular member comprising a plurality of urine inlet openings,
the proximal end portion of the elongated tubular member having a drainage outlet,
the lumen of the elongated tubular member providing fluid communication between the plurality of urine inlet openings and the drainage outlet,
at least a part of the distal end portion being preconfigured to be reversibly transformable between a linear state for passage through the urethra when inserted there through and a preconfigured coiled state for anchored receipt within the bladder in the use position, the preconfigured coiled state of the distal end portion forming a spiral emanating from a point (P) on the longitudinal axis (L) of the elongated tubular member and the spiral extending in at least a first (x) and a second direction (y), the second direction being orthogonal to the first direction and each of the first and second direction (x, y) being axially transverse to the longitudinal axis (L) of the elongated tubular member, the spiral comprising an interior spiral section facing inwards the spiral and an exterior spiral section facing outwards away from the spiral, characterized in that the plurality of urine inlet openings are provided on the interior spiral section and urine inlet openings are absent on the exterior spiral section.
